(19) United States
(12) Patent Application Publication
(10) Pub. No.: US 2022/0268772 A1
(43) Pub. Date: Aug. 25, 2022

(54) Mass Spectrometry-Based Detection Of Mycotoxins In Meat

(71) Applicants: Firoz Anwar, Jeddah (SA); Maged Al Ansari, Jeddah (SA); Fahad A. Al Abbasi, Jeddah (SA); Vikas Kumar, Prayagraj (IN)

(72) Inventors: Firoz Anwar, Jeddah (SA); Maged Al Ansari, Jeddah (SA); Fahad A. Al Abbasi, Jeddah (SA); Vikas Kumar, Prayagraj (IN)

(21) Appl. No.: 17/663,293

(22) Filed: May 13, 2022

(51) Int. Cl. G01N 33/569; G01N 30/72

(52) U.S. Cl. CPC G01N 33/56961; G01N 30/7233; G01N 2333/37; G01N 2030/027

(57) ABSTRACT

A method (100) for detecting mycotoxins in organic material, wherein the method (100) comprises of: storing about 2-5 grams of organic material in a temperature less than room temperature; adding a solvent Acetonitrile in the stored organic material to form a first mixture, wherein the first mixture is transferred to a 40-60 ml tubes, centrifuged and shaken for a defined interval; adding 0.5-3 ml of supernatant, 80-120 mg of cyclo-18-carbon (C18) and 200-400 mg of Magnesium sulfate to the first mixture to form a second mixture; and filtering the formed second mixture using a filter syringe to obtain a filtrate, wherein the mycotoxins are detected from the obtained filtrate using Ultra High-Performance Liquid Chromatography (UHPLC) coupled with a mass spectrometer.

### TECHNICAL FIELD

The present disclosure relates to a field of detection of mycotoxins. More particularly, the present disclosure relates to mass spectrometry-based detection of mycotoxins in meat.

### BACKGROUND

Fungal mycotoxins are the secondary metabolite and are harmful to both plants and animals including humans. Numerous mycotoxins have been discovered including a major class of aflatoxins produced by multiple species of Aspergillus. Common aflatoxins present and isolated from feeds and food comprises aflatoxins B1, B2, G1, G2, while M1 and M2 from milk of animal and humans.

Repeated exposure to mycotoxins is related from tumor generation to instant death. Serious manifestations occurs on interference with immune response, putting the patient to higher risk of infectious disease. Exposure to mycotoxins in humans is from ingestion of contaminated food, contact and inhalation. Public health threat or risk of foodborne disease posed by mycotoxins, led the establishment of rules and regulation intended for export or import by countries, thus limiting or narrowing the health risk of consumers. The import is assisted with multiple process including cold storage, variation in temperature and handling, humidity and health condition of the workers apart from the quality of meat.

There is increase demand of meat and meat products globally during last five decades, both in develop and developing countries. Steady increase in local population and expatriate also increased the demand from 10 kg in 1961 to 50 kg/capita/year in 2019. High import and consumption of meat make a challenging task for health authorities and food scientist to safeguard consumer health and healthy society. Contaminations of meat can results from unhygienic slaughtering, handling and processing conditions, operators' hand, unsanitary abattoir or inherent micro flora in animals along with its presence in normal tissue, air, and environment. Much of the foodborne illness are preventable and is growing public health concern. There is no exact data with Centre for disease control (CDC) that gives current burden of foodborne illness in KSA, the limited available report estimate it to be 60% from food prepared in the restaurants. Health of society, depends upon the food consumed by final consumer, it needs to be safe, processed under good hygiene from farm animal to all the production chain. The aim of this study was to determine the concentration of level of various mycotoxins including aflatoxins B1, B2, G1, G2 M1 and M2 in imported burger meat by LCMS/MS and their tentative role on apoptosis of cardiac myocytes.

U.S. Pat. No. 9,551,616B2 discloses about an approach to noninvasively and remotely detect the presence, location, and/or quantity of a target substance in a scene via a spectral imaging system comprising a spectral filter array and image capture array. For a chosen target substance, a spectral filter array is provided that is sensitive to selected wavelengths characterizing the electromagnetic spectrum of the target substance. Elements of the image capture array are optically aligned with elements of the spectral filter array to simultaneously capture spectrally filtered images. These filtered images identify the spectrum of the target substance. Program instructions analyze the acquired images to compute information about the target substance throughout the scene. A color-coded output image may be displayed on a smartphone or computing device to indicate spatial and quantitative information about the detected target substance. The system desirably includes a library of interchangeable spectral filter arrays, each sensitive to one or more target substances.

However, the above mentioned prior art discloses about complex devices and methods for detecting the target substance which is preferably a mycotoxins.

Therefore, there exists a need to propose a similar organic composition and method for detection of mycotoxins in meat.

The technical advancements disclosed by the present disclosure overcomes the limitations and disadvantages of existing and conventional systems and methods.

### BRIEF SUMMARY

The present disclosure generally relates to detection of mycotoxins in meat.

An object of the present disclosure is to mass spectrometry-based detection of mycotoxins in meat, and

Another object of the present disclosure is to use of organic materials for detecting mycotoxins.

In an embodiment, a composition for detecting mycotoxins in an organic material, wherein the composition comprises of:

2-5 grams of the organic material;
solvent Acetonitrile;
0.5-3 ml of a supernatant;
80-120 mg of a cyclo-18-carbon (C18); and
200-400 mg of a Magnesium sulfate.

In an embodiment, a method for detecting mycotoxins in organic material, wherein the method comprises of:

storing about 2-5 grams of organic material in a temperature less than room temperature; adding a solvent Acetonitrile in the stored organic material to form a first mixture, wherein the first mixture is transferred to a 40-60 ml tubes, centrifuged and shaken for a defined interval;
adding 0.5-3 ml of supernatant, 80-120 mg of cyclo-18-carbon (C18) and 200-400 mg of Magnesium sulfate to the first mixture to form a second mixture; and
filtering the formed second mixture using a filter syringe to obtain a filtrate, wherein the mycotoxins are detected from the obtained filtrate using Ultra High-Performance Liquid Chromatography (UHPLC) coupled with a mass spectrometer.

In an embodiment, the samples are collected from the organic material and stored at a temperature of 3-6°c.

In an embodiment, the first mixture is shaken with a multi vertical rotating agitator for 1-5 min at high speed, wherein the first mixture is centrifuge at 3500 rpm for 3-8 minutes.

In an embodiment, the second mixture is shaken for 0.5-2 min followed by centrifugation with 10000 rpm for 3-7 min.

In an embodiment, the filter syringe 0.45 μm polytetrafluroethylene (PTFE).

To further clarify the advantages and features of the present disclosure, a more particular description of the disclosure will be rendered by reference to specific embodiments thereof, which is illustrated in the appended drawings. It is appreciated that these drawings depict only typical embodiments of the disclosure and are therefore not to be considered limiting of its scope. The disclosure will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a method for detecting mycotoxins in organic material,

FIG. 2 illustrates a graphical representation of Pie diagram with mycotoxin concentration detected and non-detected samples,

FIG. 3 illustrates a graphical representation of Mycotoxin proportion and percentage in analyzed sample of burger meat from commercial outlets, Aflatoxins B1, B2, G1, G2, as 50%, 33%, 44% and 38.88% respectively, and

FIGS. 4 and 5 illustrates a graphical representation to demonstrate the presence of various mycotoxin alone or in co occurrence in the analyzed samples.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the disclosure, reference will now be made to the embodiment illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this disclosure belongs. The system, methods, and examples provided herein are illustrative only and not intended to be limiting.

Embodiments of the present disclosure will be described below in detail with reference to the accompanying drawings.

A composition for detecting mycotoxins in an organic material, wherein the composition comprises of:

2-5 grams of the organic material;
solvent Acetonitrile;
0.5-3 ml of a supernatant;
80-120 mg of a cyclo-18-carbon (C18); and
200-400 mg of a Magnesium sulfate.

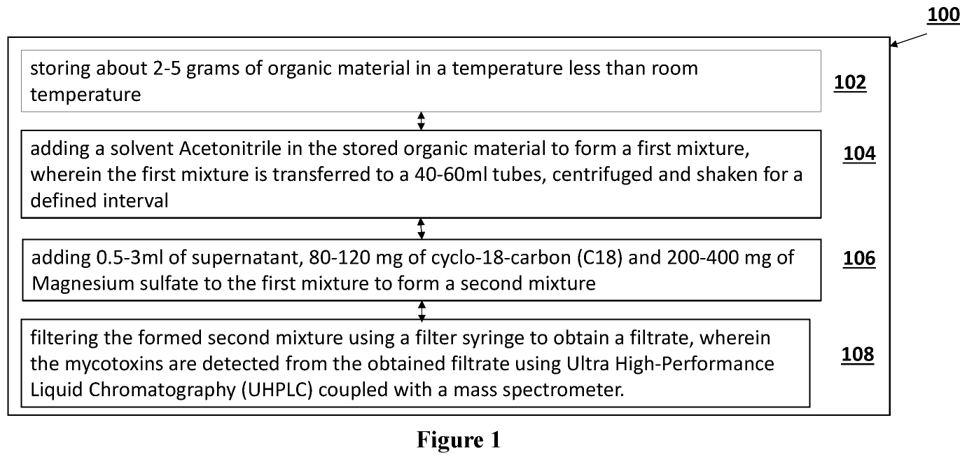

FIG. 1 illustrates a method (100) for detecting mycotoxins in organic material, wherein the method (100) comprises of:

Step (102) discloses about storing about 2-5 grams of organic material in a temperature less than room temperature, wherein the samples are collected from the organic material and stored at a temperature of 3-6° c.;

Step (104) discloses about adding a solvent Acetonitrile in the stored organic material to form a first mixture, wherein the first mixture is transferred to a 40-60 ml tubes, centrifuged and shaken for a defined interval, wherein the first mixture is shaken with a multi vertical rotating agitator for 1-5 min at high speed, wherein the first mixture is centrifuge at 3500 rpm for 3-8 minutes;

Step (106) discloses about adding 0.5-3 ml of supernatant, 80-120 mg of cyclo-18-carbon (C18) and 200-400 mg of Magnesium sulfate to the first mixture to form a second mixture, wherein the second mixture is shaken for 0.5-2 min followed by centrifugation with 10000 rpm for 3-7 min; and

Step (108) discloses about filtering the formed second mixture using a filter syringe to obtain a filtrate, wherein the mycotoxins are detected from the obtained filtrate using Ultra High-Performance Liquid Chromatography (UHPLC) coupled with a mass spectrometer, wherein the filter syringe 0.45 μm polytetrafluroethylene (PTFE).

The detection and confirmation of all mycotoxins in given samples is made by Ultra High-Performance Liquid Chromatography (UHPLC, Agilent 1290) coupled with a mass spectrometer (Sciex Triple Quad 5500) with quantification by external standard for external calibration with standard conditions. Briefly, the working conditions of UHPLC is carried out using Kinetex 2.6 μm C18 100×2.1 mm ID column (Phenomenex). Temperature of column maintained at 40° C., with a 5 μL of injection. The gradient program for the mobile phase A: 5 mM Ammonium Acetate in water and mobile phase B: Methanol was started at 95% A (0.01 min), 5% A (2 min), 95% A (3.5 min) up to 9.0 min with a flow rate 0.4 mL/min. The operating conditions for MS/MS included spectrometer operated in electrospray ionization (ESI+) along with MRM transition for quantity and quality. The limits of mass spectrometer where curtain gas was 20 ml/min, collision gas 7 ml/min, ion spray voltage 5500, nebulizer gas (gl) is 50 ml/min, evaporation gas is 50 ml/min.

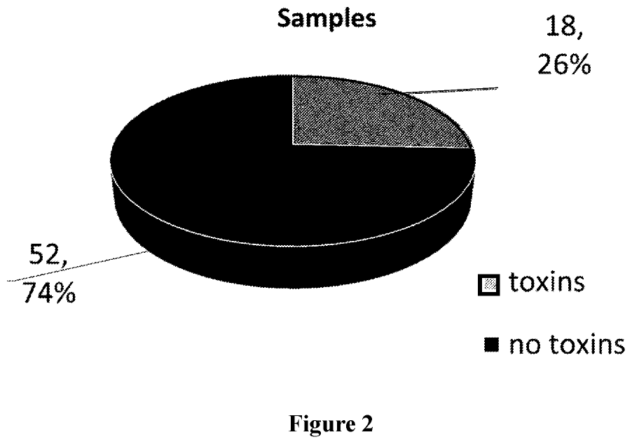

FIG. 2 illustrates a graphical representation of Pie diagram with mycotoxin concentration detected and non-detected samples. It is observed 26% (18 sample) were positive for various mycotoxin.

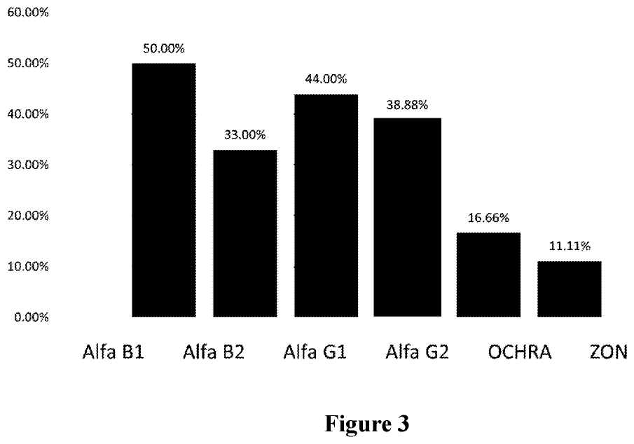

FIG. 3 illustrates a graphical representation of Mycotoxin proportion and percentage in analyzed sample of burger meat from commercial outlets, Aflatoxins B1, B2, G1, G2, as 50%, 33%, 44% and 38.88% respectively. Ocra A 16.66% and Zon 11.11%. by Triple Quad 5500. Most frequent mycotoxins proportion in the analyzed samples wasaflatoxin (AF) B1 (50%) followed by AFG1(44%), AFG2 (38.8%), AFB2 (33%) respectively Ochratoxin (Ocra A) and Zearalenone (Zon) are least among all with 16.66 and 11.11%.

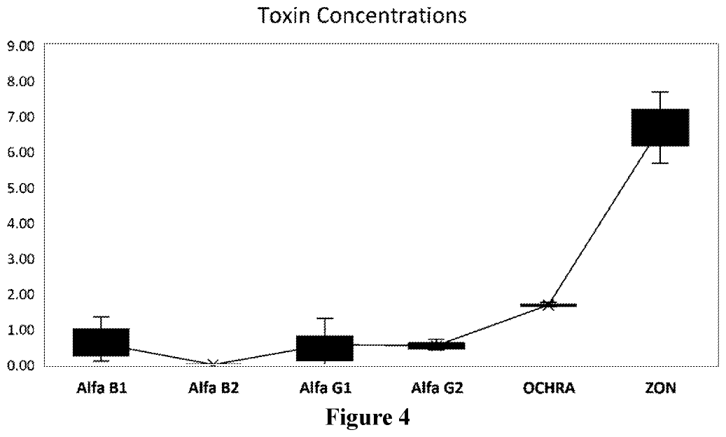

FIG. 4 illustrates a graphical representation to demonstrate the presence of various mycotoxin alone or in co occurrence in the analyzed samples.

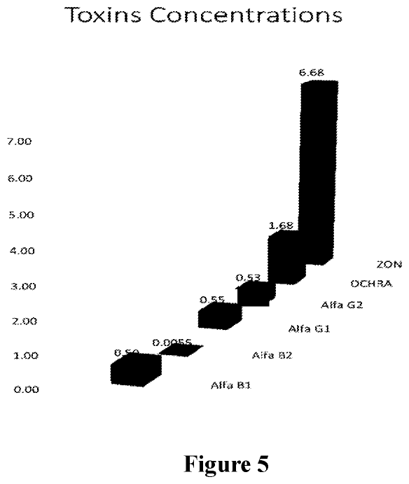

FIG. 4 demonstrates the Mycotoxin concentration in ppb for, Aflatoxins B1, B2, G1, G2, Ocra A and Zon by Triple Quad 5500 and FIG. 5 demonstrates Mycotoxin Concentration of Aflatoxins B1, B2, G1, G2 and Ocra A and Z on in detected sample with respect to ppb and percentage in analyzed sample of burger meat from commercial outlets. Eleven samples were containing more than one mycotoxin the most common aflatoxin B1 followed by aflatoxin G2. However, the highest concentration was 7.69 ppb of Zon in sample number 15. The variation in concentration is another important factor necessary for severity of the apoptosis in the humans.

Isolated mycotoxins like AFB1, B2, G1 and G2 binds to guanine and forms a DNA adduct after metabolic activation in liver, making them one of the most potent toxins responsible for carcinogenic, mutagenic, nephrotoxic, liver diseases, immunosuppressive, and hemorrhages activity. Among these aflatoxin (AF) B1 is the most studied and known to initiate apoptosis in liver via death receptor pathway. However, AFB1 induces damage to mitochondria in cardiomyocytes, promotes apoptosis and regulates the expression of apoptosis related proteins thus responsible for cardio toxicity leading to cardiomyocytes deaths. Further the increase urea, creatinine and reduction in sodium concentration in plasma by AFB1 may also contribute to damage of heart cells. Further AFB1 can induces apoptosis through interaction of p53 with Bax and Bcl-2, that can trigger caspase dependent apoptosis at mitochondrial level by impairing AMPK/mTOR-mediated autophagy flux pathway. AFB2 induced apoptosis is via activation of mitochondrial pathway through reactive oxygen species (ROS) triggering and down regulation of Bax in mitochondria, resulting in release of cyt c in cytosol, subsequently activating caspase—and 3 with cleavage of PARP. AFB2 activates PTEN and suppresses PI3K/AkT/mTOR signaling pathway.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

We claim:

1. A composition for detecting mycotoxins in an organic material, wherein the composition comprises of:
2-5 grams of the organic material;
solvent Acetonitrile;
0.5-3 ml of a supernatant;
80-120 mg of a cyclo-18-carbon (C18); and
200-400 mg of a Magnesium sulfate.

2. A method (100) for detecting mycotoxins in organic material, wherein the method (100) comprises of:
storing about 2-5 grams of organic material in a temperature less than room temperature;
adding a solvent Acetonitrile in the stored organic material to form a first mixture, wherein the first mixture is transferred to a 40-60 ml tubes, centrifuged and shaken for a defined interval;
adding 0.5-3 ml of supernatant, 80-120 mg of cyclo-18-carbon (C18) and 200-400 mg of Magnesium sulfate to the first mixture to form a second mixture; and
filtering the formed second mixture using a filter syringe to obtain a filtrate, wherein the mycotoxins are detected from the obtained filtrate using Ultra High-Performance Liquid Chromatography (UHPLC) coupled with a mass spectrometer.

3. The method as claimed in claim 2, wherein the samples are collected from the organic material and stored at a temperature of 3-6° c.

4. The method as claimed in claim 2, wherein the first mixture is shaken with a multi vertical rotating agitator for 1-5 min at high speed, wherein the first mixture is centrifuge at 3500 rpm for 3-8 minutes.

5. The method as claimed in claim 2, wherein the second mixture is shaken for 0.5-2 min followed by centrifugation with 10000 rpm for 3-7 min.

6. The method as claimed in claim 2, wherein the filter syringe 0.45 μm polytetrafluroethylene (PTFE).
